(19) United States
(12) Patent Application Publication
(10) Pub. No.: US 2024/0387023 A1
(43) Pub. Date: Nov. 21, 2024

(54) SYSTEM AND METHODS FOR IDENTIFYING EMERGENCY DEPARTMENT FREQUENT FLYERS

(71) Applicant: Optum, Inc., Minnetonka, MN (US)

(72) Inventor: Beata Konczewski, Rolling Meadows, IL (US)

(21) Appl. No.: 18/319,180

(22) Filed: May 17, 2023

(51) Int. Cl. G16H 40/20; G16H 10/60

(52) U.S. Cl. CPC G16H 40/20; G16H 10/60

(57) ABSTRACT

A system and methods for detecting patients who qualify as emergency department frequent flyers are disclosed. An emergency department frequent flyer is defined as a patient with more than a threshold number of visits to an emergency department of one or more medical facilities within a time period. The disclosed method generally includes: receiving an electronic healthcare claim; obtaining historical healthcare claims data from an electronic record of a patient associated with the electronic healthcare claim; determining whether the patient qualifies as an emergency department frequent flyer based on the electronic healthcare claim and the historical healthcare claims data; and initiating responsive actions if it is determined that the patient qualifies as an emergency department frequent to include: i) generating a flag for the patient, ii) assigning a frequent flyer category to the patient, and iii) modifying the electronic record to include the flag and category.

### BACKGROUND

The emergency department (ED) or emergency room (ER) is a critical component of the healthcare system, providing life-saving care to patients with urgent and emergent medical needs. While ED visits are sometimes unavoidable, a subset of patients referred to as “frequent flyers” or “super-users” disproportionately utilize emergency services. These patients may have multiple chronic conditions, mental health or substance abuse issues, or lack access to primary care, resulting in frequent visits to the ED. Frequent ED use can be costly to patients, healthcare providers, and payors. It is estimated that up to 5% of patients account for 20% of all ED visits, and these patients have been shown to have higher rates of hospitalization, readmission, and mortality compared to non-frequent users. Additionally, the burden of frequent ED visits can strain hospital resources and divert attention away from other patients in need of emergency care.

### SUMMARY

One implementation of the present disclosure is a method of detecting patients who qualify as emergency department frequent flyers, the method including: receiving an electronic healthcare claim; obtaining historical healthcare claims data from an electronic record of a patient associated with the electronic healthcare claim; determining whether the patient qualifies as an emergency department frequent flyer based on the electronic healthcare claim and the historical healthcare claims data, wherein an emergency department frequent flyer is defined as a patient with more than a threshold number of visits to an emergency department of one or more medical facilities within a time period; and initiating responsive actions if it is determined that the patient qualifies as an emergency department frequent flyer, wherein the responsive actions include: i) generating a flag for the patient that identifies the patient as an emergency department frequent flyer, ii) assigning a frequent flyer category to the patient based on a procedure code or a diagnosis code contained in the electronic healthcare claim, and iii) modifying the electronic record of the patient to include the flag and the frequent flyer category.

Another implementation of the present disclosure is a system for detecting patients who qualify as emergency department frequent flyers, the system including: one or more processors; and memory having instructions stored thereon that, when executed by the one or more processors, cause the system to: receive an electronic healthcare claim; obtain historical healthcare claims data from an electronic record of a patient associated with the electronic healthcare claim; determine whether the patient qualifies as an emergency department frequent flyer based on the electronic healthcare claim and the historical healthcare claims data, wherein an emergency department frequent flyer is defined as a patient with more than a threshold number of visits to an emergency department of one or more medical facilities within a time period; and initiate responsive actions if it is determined that the patient qualifies as an emergency department frequent flyer, wherein the responsive actions include: i) generating a flag for the patient that identifies the patient as an emergency department frequent flyer, ii) assigning a frequent flyer category to the patient based on a procedure code or a diagnosis code contained in the electronic healthcare claim, and iii) modifying the electronic record of the patient to include the flag and the frequent flyer category.

Yet another implementation of the present disclosure is a non-transitory computer readable medium having instructions stored thereon that, when executed by one or more processors, cause a device to: receive an electronic healthcare claim; obtain historical healthcare claims data from an electronic record of a patient associated with the electronic healthcare claim; determine whether the patient qualifies as an emergency department frequent flyer based on the electronic healthcare claim and the historical healthcare claims data, wherein an emergency department frequent flyer is defined as a patient with more than a threshold number of visits to an emergency department of one or more medical facilities within a time period; and initiate responsive actions if it is determined that the patient qualifies as an emergency department frequent flyer, wherein the responsive actions include: i) generating a flag for the patient that identifies the patient as an emergency department frequent flyer, ii) assigning a frequent flyer category to the patient based on a procedure code or a diagnosis code contained in the electronic healthcare claim, and iii) modifying the electronic record of the patient to include the flag and the frequent flyer category.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

FIG. 1 is a block diagram of a communication topology for evaluating electronic healthcare claims to identify “frequent flyers”, according to some implementations.

FIG. 2 is a block diagram of the frequent flyer evaluation system of FIG. 1, according to some implementations.

FIG. 3 is a flow diagram of a process for identifying emergency department frequent flyers, according to some implementations.

FIG. 4 is a flow diagram of a process for predicting whether a patient is at risk of becoming a frequent flyer, according to some implementations.

Various objects, aspects, and features of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

### DETAILED DESCRIPTION

Referring generally to the figures, a system and methods for identifying emergency department (ED) “frequent flyers” are shown, according to various implementations. As described herein, an ED frequent flyer—also referred to as a “frequent flyer” or “ED super-user”—is defined as a patient with more than a threshold number of visits to an ED of one or more medical facilities within a time period. Generally, the threshold number of visits and corresponding time period that define a frequent flyer is either four visits within twelve months or three visits within three months; however, it should be appreciated that other criteria may be used to define a “frequent flyer.” It should also be appreciated that, throughout the present disclosure, ED and emergency room (ER) may be used interchangeably and generally refer to a medical treatment facility that specializes in acute patient care without a prior appointment.

The disclosed system and methods are generally able to quickly and efficiently identify ED frequent flyers by evaluating electronic healthcare claims against corresponding historical and payor-agnostic claims data for one or more patients. For example, when an electronic healthcare claim is received, the disclosed system is configured to retrieve or otherwise reference historical claims data for the associated patient to determine whether the patient qualifies as a frequent flyer. If so, various responsive actions can be automatically initiated, including but not limited to flagging the patient as a frequent flyer and optionally generating a care management path for the patient. In this manner, the underlying reasons for the patient's frequent ED visits (e.g., alcohol abuse) can be addressed, e.g., by connecting the patient with an appropriate medical professional. Providing the right kind of care for frequent flyers can not only save providers and payors money and decrease instances of ED overpopulation but can also lead to improved patient outcomes.

### Overview

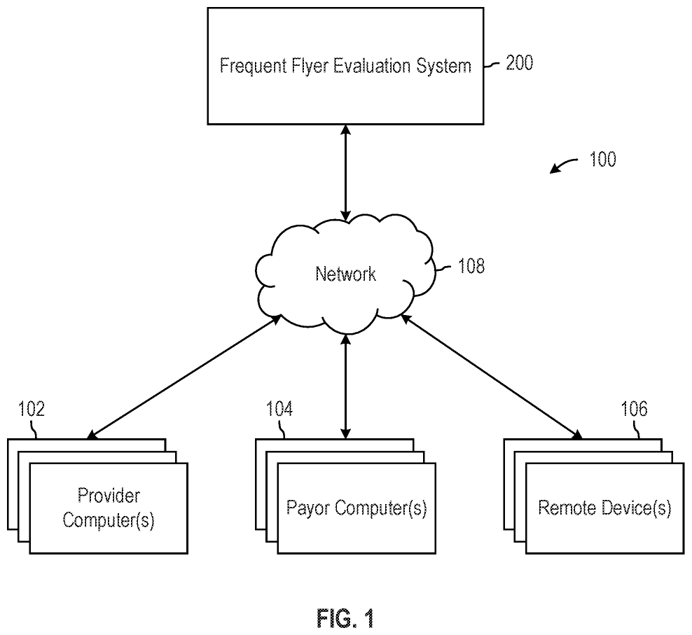

Referring to FIG. 1, a block diagram of an example communication topology 100 for evaluating electronic healthcare claims to identify frequent flyers is shown, according to some implementations. Generally, communication topology 100 includes one or more computing devices that can communicate with (e.g., send data to and/or receive data from) a frequent flyer evaluation system 200 which, as discussed in detail below, is configured to identify patients that qualify as frequent flyers. In the example shown, communication topology 100 can include one or more provider computers 102, one or more payor computers 104, and one or more other remote devices 106. Provider computers 102 generally include one or more computing devices or systems (e.g., desktop or laptop computers, servers, workstations, etc.) that are operated by healthcare providers (e.g., physicians, hospitals, etc.) or other parties that submit electronic healthcare claims. Similarly, payor computers 104 generally include one or more computing devices or systems (e.g., desktop or laptop computers, servers, workstations, etc.) that are operated by a party that provides payment for electronic healthcare claims (e.g., healthcare insurance companies). Remote devices 106 can generally include one or more additional computing devices or systems, e.g., other than those operated by healthcare providers or payors, as described in greater detail below. For example, remote devices 106 may be operated by various medical billing companies.

It should be understood that, as described herein, provider computers 102, payor computers 104, and remote devices 106 may each include any number of individual computing devices or computing systems and may be associated with any number of users. For example, provider computers 102 may refer to multiple different computing devices associated with multiple different healthcare providers, while payor computers 104 may refer to a single computing device associated with a single healthcare payor, or vice versa. Likewise, any of provider computers 102, payor computers 104, or remote devices 106 may be optional. For example, in some implementations, only provider computers 102 may interact with system 200. Thus, it should be appreciated that the number and arrangement of computing devices that can communicate with system 200 is not intended to be limiting.

In some implementations, provider computers 102, payor computers 104, and/or remote devices 106 communicate with system 200 via a network 108. As described herein, network 108 may be any suitable network to facilitate the transmission of data between components of communication topology 100. In particular, network 108 may facilitate the transmission of data, such as electronic healthcare claims, from/to system 200. Examples of suitable networks include, but are not limited to, local area network (LANs), wide area networks (WANs), virtual private networks (VPNs), and the like. In one example, network 108 is the Internet (e.g., a WAN). However, it should be appreciated that, in some implementations, any of provider computers 102, payor computers 104, and/or remote devices 106 may communicate directly with system 200 (e.g., without network 108). Additionally, network 108 may generally represent more than one type of network. For example, one of provider computers 102 may communicate with system 200 via a first network type while another one of provider computers 102 communicates with system 200 via a second network type.

Electronic healthcare claims typically contain information about a patient, a healthcare provider, healthcare services rendered, and costs associated with those services, as generally known to those of ordinary skill in the art. In most cases, electronic healthcare claims are submitted by healthcare providers (e.g., doctors, hospitals, clinics, and the like), or by medical billing companies on behalf of healthcare providers, to healthcare payors (e.g., health insurance companies, government health programs, etc.) for reimbursement of healthcare services. For example, a healthcare provider or medical billing company may generate an electronic healthcare claim on a local device (e.g., one of provider computers 102), which is then transmitted to a payor or intermediary party for adjudication and payment. In some implementations, electronic healthcare claims are submitted as Electronic Data Interchange (EDI) 837 (“Health Care Claim”) transactions, although other suitable formats may be used.

In some implementations, electronic healthcare claims are transmitted directly from the submitting party to an appropriate healthcare payor. For example, a healthcare provider may transmit the electronic healthcare claim directly to a patient's insurance company. In such implementations, an electronic healthcare claim can be transmitted directly from one of provider computers 102—or one of remote devices 106, if operated by a medical billing company—to one of payor computers 104. In some such implementations, the receiving party (e.g., the healthcare payor) can store the electronic healthcare claim locally and/or on their own computing system (e.g., a server). For example, upon receipt, a healthcare payor may store an electronic healthcare claim in a secure local or remote database for later processing. In some implementations, electronic healthcare claims are instead transmitted to a remote database (e.g., hosted on a cloud server) that can be remotely accessed by the healthcare payor. In some implementations, electronic healthcare claims are transmitted to an intermediary party and/or intermediary computing system (e.g., one of remote devices 106) for preprocessing and/or storage, prior to receipt by the healthcare payor.

System 200, as mentioned above, is generally configured to identify ED frequent flyers based in part on submitted electronic healthcare claims. In this regard, system 200 may be configured to obtain electronic healthcare claims from any of provider computers 102. payor computers 104, or certain ones of remote devices 106 (e.g., remote databases, medical billing company computers, etc.). In some implementations, system 200 receives electronic healthcare claims directly from healthcare providers via provider computers 102. For example, healthcare providers may submit electronic healthcare claims to system 200 before they are submitted to a healthcare payor and/or system 200 may be configured to intercept electronic healthcare claims as they are transmitted to a healthcare payor. In some such implementations, system 200 may be configured to forward a received electronic healthcare claim to an appropriate payor after evaluating against frequent flyer criteria. In other implementations, system 200 can receive electronic healthcare claims from healthcare payors, e.g., via payor computers 104. For example, healthcare providers may transmit individual claims or batches of claims to system 200 for evaluation. In yet other implementations, system 200 can access one or more remote databases, e.g., separate from or associated with any number of healthcare providers or payors, to retrieve electronic healthcare claims.

To this point, system 200 is generally remotely accessible to a number of different healthcare providers, medical billing companies, healthcare payors, and other relevant parties. For example, as described in greater detail below, system 200 may include an application programming interface (API) that facilitates communication with outside computing systems and devices such that system 200 is remotely accessible, e.g., via the Internet. Thus, system 200 can be, or can be hosted on, any suitable computing device. For example, system 200 can be hosted on a cloud server or, more generally, any server configured for cloud-based computing. Alternatively, in some implementations, the functionality of system 200 as described herein can be implemented by any of provider computers 102, payor computers 104, or remote devices 106. For example, system 200 and/or the functionality thereof may be implemented by one of payor computers 104 such that the associated healthcare payor can directly evaluate electronic healthcare claims to identify frequent flyers.

### ED Frequent Flyer Identification

Generally, upon obtaining an electronic healthcare claim, system 200 is configured to: identify the associated patient; extract a subset of the claim data relevant to determining whether the patient qualifies as a frequent flyer; retrieve or otherwise access historical healthcare claims data for the patient, e.g., from a database; and evaluate the extracted electronic healthcare claim data and the patient's historical healthcare claims data to determine if the patient qualifies as a frequent flyer. As discussed in greater detail below, system 200 may initiate various responsive actions if it is determined that the patient qualifies as a frequent flyer, including but not limited to flagging the patient and/or adding the patient to a list of frequent flyers, generating a care path for the patient, and/or alerting a user (e.g., a user of one of payor computers 104) that the patient qualifies as a frequent flyer. In addition, in some implementations, system 200 can predict whether a patient is at risk of becoming a frequent flyer, even before the patient fully qualifies (e.g., by meeting a set of frequent flyer criteria). Additional details of system 200 are discussed below.

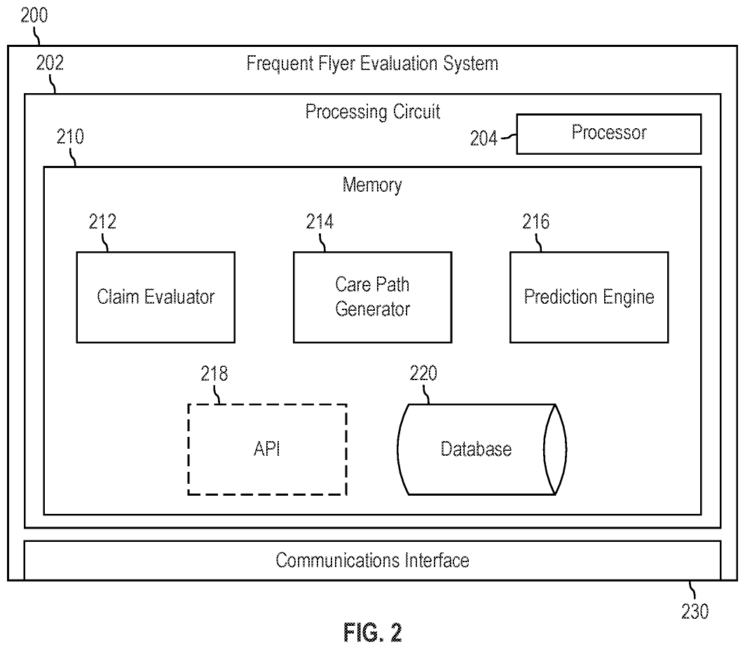

Referring now to FIG. 2, a detailed block diagram of system 200 is shown, according to some implementations. System 200 generally includes a processing circuit 202 that includes a processor 204 and a memory 210. Processor 204 can be a general-purpose processor, an application-specific integrated circuit (ASIC), one or more field programmable gate arrays (FPGAs), a group of processing components, or other suitable electronic processing components. In some implementations, processor 204 is configured to execute program code stored on memory 210 to cause system 200 to perform one or more operations.

Memory 210 can include one or more devices (e.g., memory units, memory devices, storage devices, etc.) for storing data and/or computer code for completing and/or facilitating the various processes described in the present disclosure. In some implementations, memory 210 includes tangible, computer-readable media that stores code or instructions executable by processor 204. Tangible, computer-readable media refers to any media that is capable of providing data that causes system 200 to operate in a particular fashion. Example tangible, computer-readable media may include, but is not limited to, volatile media, non-volatile media, removable media and non-removable media implemented in any method or technology for storage of information such as computer readable instructions, data structures, program modules or other data. Accordingly, memory 210 can include random access memory (RAM), read-only memory (ROM), hard drive storage, temporary storage, non-volatile memory, flash memory, optical memory, or any other suitable memory for storing software objects and/or computer instructions. Memory 210 can include database components, object code components, script components, or any other type of information structure for supporting the various activities and information structures described in the present disclosure. Memory 210 can be communicably connected to processor 204, such as via processing circuit 202, and can include computer code for executing (e.g., by processor 204) one or more processes described herein.

While shown as individual components, it will be appreciated that processor 204 and/or memory 210 can be implemented using a variety of different types and quantities of processors and memory. For example, processor 204 may represent a single processing device or multiple processing devices. Similarly, memory 210 may represent a single memory device or multiple memory devices. Additionally, in some implementations, system 200 may be implemented within a single computing device (e.g., one server, one housing, etc.). In other implementations, system 200 may be distributed across multiple servers or computers (e.g., that can exist in distributed locations). For example, system 200 may include multiple distributed computing devices (e.g., multiple processors and/or memory devices) in communication with each other that collaborate to perform operations. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by two or more computers. For example, virtualization software may be employed by system 200 to provide the functionality of a number of servers that is not directly bound to the number of computers in system 200.

Memory 210 is shown to include a claim evaluator 212 configured to evaluate received electronic healthcare claims against a patient's claim history to determine if the patient qualifies as a frequent flyer. As described above, electronic healthcare claims may be obtained in several ways. In some implementations, electronic healthcare claims are received directly from healthcare providers and/or medical billers. In some such implementations, the electronic healthcare claim(s) can be forwarded to an appropriate payor (i.e., the original recipient) after the evaluation performed by system 200. In some implementations, electronic healthcare claims are intercepted as they are transmitted from healthcare providers and/or medical billers to payors. For example, electronic healthcare claims may pass through a clearinghouse that intercepts the claim and/or claim data for analysis. In some implementations, electronic healthcare claims are received from payors, e.g., during adjudication and/or during processing prior to payment. In some such implementations, a payor may transmit individual claims and/or batches of claims to system 200. In some implementations, electronic healthcare claims are stored in a database and are retrieved by system 200 for evaluation. In some such implementations, the database may be local to system 200 (e.g., shown as a database 220 in FIG. 2) or may be remote from system 200. In some implementations, after receipt/retrieval system 200 can store a local copy of electronic healthcare claims that are originally stored remotely and/or maintained by a remote device.

Generally, upon obtaining an electronic healthcare claim, claim evaluator 212 is configured to identify the associated patient and to extract a subset of the claim data relevant to determining whether the patient qualifies as an ED frequent flyer. For example, claim evaluator 212 may identify the patient based on patient information (e.g., the patient's name, an identification number, a phone number, etc.) contained within the header of the electronic healthcare claim and may extract data from the header and/or body of the claim relevant to determining whether the patient qualifies as a frequent flyer. With respect to an EDI 837 file, this may include identifying relevant claim elements and extracting the associated data. In some implementations, claim evaluator 212 is configured to extract at least a service date associated with the electronic healthcare claim or a receipt date of the electronic healthcare claim, an identifier for a medical facility at which the patient was treated, a revenue code, a procedure code or a diagnosis code, a filing indicator code, and a payor identifier, which are all elements that may be relevant to determining whether the patient is a frequent flyer. In some implementations, claim evaluator 212 includes a natural language processing (NLP) model, or another similar type of model, for evaluating free text, e.g., to extract additional details for making a frequent flyer determination. For example, claim evaluator 212 may use an NLP model to process unstructured data (e.g., text) in the patient's electronic healthcare claim.

After identifying the patient associated with the electronic healthcare claim, claim evaluator 212 can retrieve or otherwise access historical healthcare claims data for the patient, e.g., from an electronic record maintained in a database. In various implementations, the database is a local database (e.g., database 220) or a remote database. For example, in some implementations, claim evaluator 212 may retrieve historical healthcare claims data from the patient's health insurance company (e.g., a healthcare payor). Historical healthcare claims data generally includes data relating to any past electronic healthcare claims submitted for the patient, including copies of any past electronic healthcare claims. Notably, claim evaluator 212 may be configured to obtain or access historical healthcare claims data associated with a number of different healthcare providers (or medical billers) and/or healthcare payors. In this way, the frequent flyer determination made by claim evaluator 212 is payor-agnostic and can account for claims submitted by various different providers and/or to various different payors. In other words, system 200 can identify frequent flyers even among patients that visit different EDs/ERs and/or that have claims submitted to different payors, which can identify patients that may have been missed using payor-specific data. Additionally, identifying patients as frequent flyers regardless of the provider and/or payor associated with the patient can reduce occurrences of misdiagnosis, e.g., due to healthcare providers having a limited knowledge of past visits to other providers.

Using the data extracted from the received electronic healthcare claim and the obtained historical healthcare claims data for the patient, claim evaluator 212 can determine whether the patient qualifies as a frequent flyer. Generally, a frequent flyer determination is dependent, at least in part, on a service date associated with the received electronic healthcare claim. Alternatively, if a service date is unavailable, claim evaluator 212 may utilize a date that the electronic healthcare claim was received (e.g., a “receipt date”). In some implementations, claim evaluator 212 uses the service or receipt date of the electronic healthcare claim and the service/receipt dates of one or more past electronic healthcare claim in the patient's claim history to determine a number of visits, n, the patient made to an ER/ED over the last m months, where n and m are positive integers.

As mentioned above, a frequent flyer is typically defined as a patient who visits an ED four or more times in 12 months or three or more times in three months; however, other frequent flyer criteria may be used (e.g., six visits in 24 months). Accordingly, the value m may be a predefined number of months (e.g., three months, 12 months, etc.), as determined by the frequent flyer criteria, and the value of n may be determined by claim evaluator 212 based on the electronic healthcare claim and historical claims data. Claim evaluator 212 can therefore determine that a patient qualifies as a frequent flyer if n (i.e., the number of visits in m months) exceeds a threshold value (e.g., four visits in 12 months). In some implementations, claim evaluator 212 can evaluate an electronic healthcare claim against multiple different frequent flyer criteria, including different thresholds for different time periods (e.g., three visits in three months or four visits in 12 months).

In some implementations, claim evaluator 212 considers additional parameters when determining whether a patient qualifies as a frequent flyer, such as the location/provider associated with each visit, diagnosis and/or procedure codes (e.g., ICD-10 codes), and other data elements extracted from the electronic healthcare claim. In some implementations, claim evaluator 212 can consider a service location (e.g., an identifier for the healthcare provider or department where the patient was treated) and/or a revenue code, as extracted from the electronic healthcare claim, in determining whether the patient is a frequent flyer. Specifically, claim evaluator 212 may determine whether the service location and/or revenue code are associated with an ED or ER. If so, the electronic healthcare claim can be further processed, e.g., to make a frequent flyer determination. If not, claim evaluator 212 may disregard the electronic healthcare claim because it is not associated with an ED or ER visit.

In some implementations, claim evaluator 212 considers the diagnosis and/or procedure codes contained in the electronic healthcare claim, and/or the diagnosis and/or procedure codes in each historical healthcare claim, to separate qualifying events from non-qualifying events. For example, when a new electronic healthcare claim is obtained, claim evaluator 212 may first determine if the claim is associated with a non-qualifying event and, if so, may remove, disregard, or otherwise filter the claim from consideration in making a frequent flyer determination. Non-qualifying events, as described herein, are predefined events—as determined by diagnosis/procedure codes—that are not counted towards a frequent flyer determination. In other words, non-qualifying events are not counted towards the number of visits to an ED, n, for a patient when making a frequent flyer determination. Example non-qualifying events include, but are not limited to, accident-related injuries, such as broken bones, concussions, and the like, which are typically not indicative of a frequent flyer. For example, a patient that gets in multiple car accidents within a 12-month period may not be considered a frequent flyer because the patient is not willfully over-utilizing the ED.

When a patient is determined to qualify as a frequent flyer, claim evaluator 212 can automatically initiate various responsive actions. In some implementations, claim evaluator 212 initially generates a flag that identifies the patient as a frequent flyer. In some implementations, multiple flags are generated if the patient meets multiple different frequent flyer criteria. For example, if the patient has visited an ED four times in less than three months, they may be flagged twice—once for exceeding a ‘three visits in three months’ threshold and once for exceeding a ‘four visits in 12 months' threshold.’ In some implementations, the flag is an electronic record that is stored (e.g., in database 220) and/or transmitted to appropriate healthcare providers and/or payors. The flag may identify the patient (e.g., by name), indicate that the patient qualifies as a frequent flyer, and provide further details regarding the rationale for the frequent flyer determination, such as a location of service (e.g., an address of the ED the patient most recently visited), a revenue code, diagnosis and/or procedure codes, payor identifier, and optionally patient demographic information. In some implementations, rather than creating a separate record, claim evaluator 212 updates an existing electronic record for the patient. For example, claim evaluator 212 may update a patient profile to indicate that the patient is a frequent flyer, and may also include details regarding the rationale for the frequent flyer determination as discussed above.

In some implementations, claim evaluator 212 can further assign the patient to a frequent flyer “category” based on the diagnosis/procedure codes contained in the electronic healthcare claim and the patient's historical claims data. Frequent flyer categories may be predefined, in some implementations, and can be used to categorize the patient based on the reason for their qualification as a frequent flyer. Generally, as mentioned above, a principal diagnosis or procedure code is used to determine an appropriate category. For example, a patient that visits the ED three times within two months, and with each visit has a principal diagnosis code of “F10—Alcohol Abuse,” may be assigned to an “alcohol abuse” category. In some implementations, the assigned category is further included with the flag, e.g., in the generated electronic record or appended to the patient's existing electronic record. In some implementations, claim evaluator 212 determines a frequent flyer category based on a set of rules. In some such implementations, diagnosis/procedure codes are mapped to associated frequent flyer categories.

In some implementations, the assigned frequent flyer category and/or other data from the patient's electronic record (e.g., including historical claims data and the electronic healthcare claim) are used to generate a care management path. Specifically, memory 210 can include a care path generator 214 that can generate a care management path for the patient if it is determined that the patient qualifies as a frequent flyer. A “care management path.” as described herein, is a predicted healthcare path for the patient that is intended to address the underlying reason for the patient qualifying as a frequent flyer. Generally, a care management path includes one or more potential/predicted future visits with a healthcare provider, to a healthcare facility, or the like. To continue the previous example, care path generator 214 may generate a care path that includes a visit with a social worker or substance abuse counselor, a recommendation for rehabilitation, etc., for a patient that is classified as a frequent flyer for alcohol abuse.

In some implementations, care path generator 214 first determines a reason for the patient being considered a frequent flyer (e.g., based on the frequent flyer category) and then identifies a suitable, predefined care path. For example, a plurality of care paths for common frequent flyer categories (e.g., drug use, alcohol abuse, etc.) may be predefined. In some such implementations, at least a type of next visit may be defined. In other words, the care path may indicate at least a type of next visit for the patient (e.g., a visit with a counselor, a stay in a rehabilitation facility, etc.). In some implementations, care path generator 214 can consider the patient's historical claims data in making a care path determination. For example, if a patient has already seen a counselor yet is still a frequent flyer due to alcohol abuse, care path generator 214 may determine that a more appropriate next step is rehabilitation. In some implementations, care path generator 214 is configured to automatically schedule at least one subsequent visit with an appropriate healthcare professional (e.g., a counselor) based on the generated care management path. Further details of care management path generation are discussed below and in U.S. Ser. No. 17/835,104, filed on Jun. 8, 2022, which is incorporated by reference herein in its entirety.

In some implementations, claim evaluator 212 is further configured to generate an alert if it is determined that a patient qualifies as a frequent flyer. The alert may identify the patient and may indicate a reason for the patient qualifying as a frequent flyer. In some implementations, the alert includes the assigned frequent flyer category and/or includes details of the automatically generated care management path. Generally, claim evaluator 212 is configured to present the alert to a suitable user, such as a healthcare professional or a healthcare payor representative. For example, the alert may be presented via a user interface, e.g., of one of provider computers 102 or payor computers 104. In some implementations, system 200 is configured to transmit alerts to remote devices (e.g., any of provider computers 102, payor computers 104, or remote devices 106). For example, system 200 may transmit the alert to a healthcare provider's computer to prompt the healthcare provider to schedule a follow-up visit with the patient.

In some implementations, claim evaluator 212 is configured to identify one or more entities to receive the alert. For example, the healthcare provider that treated the patient and the healthcare payor to whom the claim was submitted may both be notified. In some such implementations, claim evaluator 212 may be able to automatically identify one or more recipients based on the data in the electronic healthcare claim. For example, claim evaluator 212 may identify a recipient of the claim (e.g., a healthcare payor) and may, in turn, send an alert to the intended recipient. In some implementations, claim evaluator 212 identifies a recipient for the alert based on diagnosis and/or procedure codes in the electronic healthcare claim. In some implementations, in addition to or in lieu of generating an alert, the patient is added to a “frequent flyers” list, which can include identifiers for one or more frequent flyers. In some such implementations, the frequent flyers list and/or the aforementioned flag can be used to rapidly identify frequent flyers during subsequent visits. For example, if a patient who is flagged as a frequent flyer visits an ED anytime after being flagged, the healthcare provider (e.g., the ED employees) can be instantly or near-instantly notified.

As shown, memory 210 can further include a prediction engine 216 configured to predict whether a patient is at risk of becoming a frequent flyer even before the patient qualifies as a frequent flyer based on the above-mentioned frequent flyer criteria. In particular, prediction engine 216 can include a predictive model configured to predict a likelihood that a patient may become a frequent flyer based on a received electronic healthcare claim and/or the patient's claim history. In some implementations, data from a received electronic healthcare claim and/or the patient's claim history is provided as an input to prediction engine 216, which outputs a corresponding prediction. In some implementations, prediction engine 216 includes a machine learning model, such as a classifier or neural network. For example, prediction engine 216 may include one of a logistic regression model, a Naive Bayes model, a K-nearest neighbors model, a decision tree, support vector machines (SVM), or the like, which determines a class based on input data (e.g., “frequent flyer” or “not a frequent flyer”) and, in some cases, which outputs a confidence score in the predicted class. In some implementations, prediction engine 216 simply outputs a score or value (e.g., a percentage) indicative of a likelihood that the patient qualifies as a frequent flyer in the future based on current and/or historical data.

In some implementations, the predictive model implemented by prediction engine 216 is trained using a training data set of historic healthcare claims data for a plurality of patients. Specifically, the training data set may include data extracted for past electronic healthcare claims for different patients, including service or receipt data, diagnosis and/or procedure codes, and the like. In some implementations, the predictive model is trained using known supervised learning techniques, in which case the training data set may also include an indication of whether each patient was found to be a frequent flyer. For example, a training data set that includes historic healthcare claims data for patients ‘A,’ ‘B.’ and ‘C’ may indicate that patients ‘A’ and ‘B’ were not frequent flyers, but patient ‘C’ was. Thus, the predictive model can be trained to identify patterns indicative of frequent flyers.

Communications interface 230 may facilitate communications between system 200 and any external components or devices, including any of provider computers 102, payor computers 104, and remote devices 106. Accordingly, communications interface 230 can be or can include any combination of one or more wired or wireless communications interfaces (e.g., jacks, antennas, transmitters, receivers, transceivers, wire terminals, etc.) for conducting data communications. For example, communications interface 230 may include both a wired communication interface (e.g., an Ethernet port) and a wireless communications interface (e.g., a short-range wireless transceiver). In various implementations, communications via communications interface 230 may be direct (e.g., local wired or wireless communications) or via a network (e.g., a LAN, the Internet, a cellular network, etc.), such as network 108. For example, communications interface 230 can include a Wi-Fi transceiver for communicating via a wireless communications network to the Internet.

In some implementations, communications between system 200 and any external devices/systems are further facilitated by an API 218. Generally, API 218 can be any suitable API, such as a RESTful API, a SOAP API, or the like. As will be appreciated by those of ordinary skill in the art, an API is a connectivity interface that facilitates the communication (e.g., transmission and/or receipt) of data between two or more systems and devices, e.g., by applying a set of protocols and/or rules for communication. API 218 generally allows external devices, such as provider computers 102 and payor computers 104, to communicate with system 200. For example, as mentioned above, system 200 may be hosted on a cloud server; thus, API 218 can be utilized by provider computers 102, payor computers 104, and/or remote devices 106 to send and receive data to/from system 200, including patient claims data (e.g., electronic healthcare claims and/or patient claims history).

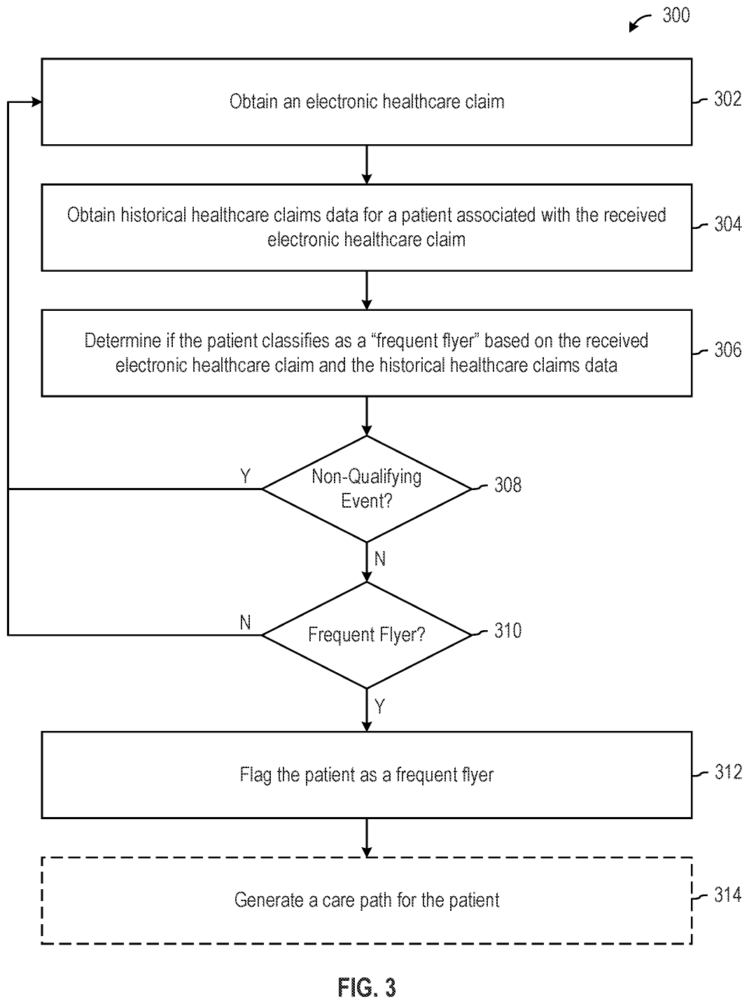

Referring now to FIG. 3, a flow diagram of a process 300 for identifying emergency department frequent flyers is shown, according to some implementations. In some implementations, process 300 is implemented by system 200, as described above. However, it should be appreciated that process 300 may be implemented by other computing devices. For example, process 300 may be implemented via non-transitory computer readable medium or may be executed (e.g., as a script) on one of provider computers 102 or payor computers 104. It will be appreciated that certain steps of process 300 may be optional and, in some implementations, process 300 may be implemented using less than all of the steps. It will also be appreciated that the order of steps shown in FIG. 3 is not intended to be limiting.

At step 302, an electronic healthcare claim is obtained. As described above, the electronic healthcare claim may be received directly from a healthcare provider and/or a medical biller or a healthcare payor. In some implementations, the electronic healthcare claim is intercepted as it is transmitted from a healthcare provider and/or medical biller to a healthcare payor. For example, electronic healthcare claims may pass through a clearinghouse that intercepts the claim. In some implementations, the electronic healthcare claim is retrieved from a database. For example, the electronic healthcare claim may be retrieved from a database associated with a healthcare payor or another remote database. In some implementations, the electronic healthcare claim is one of a batch of electronic healthcare claims that are intended to be processed together (e.g., sequentially). For example, a healthcare payor may transmit a batch of electronic healthcare claims to system 200 for evaluation. In some such implementations, it will be appreciated that process 300 may be repeated to evaluate each claim of the batch of claims. In some implementations, the electronic healthcare claim is maintained by a healthcare provider or payor, and process 300, as described herein, is implemented locally on the healthcare provider or payor's computing system.

At step 304, historical healthcare claims data for the patient is obtained. Specifically, in some implementations, a record of the patient's past electronic healthcare claims is obtained. Generally, the historical healthcare claims data includes copies of, or data extracted from, any number of past electronic healthcare claims associated with the patient. In some implementations, the historical healthcare claims data may include at least data associated with any healthcare claims from the past m months, where m is a number of months defined by frequent flyer criteria, as mentioned above. For example, if a frequent flyer is defined as a patient with more than four ED visits in 12 months, at least the last 12 months of healthcare claims data may be obtained for the patient. In various implementations, historical healthcare claims data is obtained from healthcare payors or from a separate database. For example, healthcare payors may maintain records of past healthcare claims which can be retrieved or otherwise referenced. In some implementations, the historical healthcare claims data is maintained by another remote database (e.g., database 220). For example, system 200 may maintain a database of historical healthcare claims data associated with multiple different healthcare providers, payors, and/or patients.

At step 306, a determination of whether the patient qualifies as a frequent flyer is made. In particular, the electronic healthcare claim and historical healthcare claims data is compared to frequent flyer criteria to determine if the patient qualifies as a frequent flyer. In some implementations, this includes first extracting relevant data from the electronic healthcare claim and/or historical healthcare claims data, such as service and/or receipt data, diagnosis and/or procedure codes, service location (e.g., an address or identifier of the medical facility the patient was treated at), and revenue code. Then, the extracted data may be compared to frequent flyer criteria. Generally, the frequent flyer criteria define a threshold number of visits to an ED/ER within a time period (e.g., four visits in 12 months). In some implementations, as mentioned above, the frequent flyer criteria may include multiple different thresholds for different time periods (e.g., four visits in 12 months or three visits in three months).

In some implementations, claim evaluator 212 considers additional parameters when determining whether a patient qualifies as a frequent flyer, such as the location/provider associated with each visit, diagnosis and/or procedure codes (e.g., ICD-10 codes), and other data elements extracted from the electronic healthcare claim. In some implementations, claim evaluator 212 can consider a service location (e.g., an identifier for the healthcare provider or department where the patient was treated) and/or a revenue code, as extracted from the electronic healthcare claim, in determining whether the patient is a frequent flyer. Specifically, claim evaluator 212 may determine whether the service location and/or revenue code are associated with an ED or ER. If so, the electronic healthcare claim can be further processed, e.g., to make a frequent flyer determination. If not, claim evaluator 212 may disregard the electronic healthcare claim because it is not associated with an ED or ER visit.

As mentioned above, in some implementations, diagnosis and/or procedure codes can be further considered to separate qualifying events from non-qualifying events (step 308). Generally, non-qualifying events are visits that are associated with an event that does not qualify the patient for frequent flyer status. In other words, non-qualifying events are not counted towards the number of visits to an ED for a patient when making a frequent flyer determination. As an example, a patient that visits the ED three times in three months for broken bones may not necessarily qualify as a frequent flyer, as broken bones are generally associated with an accident and not over-use of the ED by the patient. In some implementations, a principal diagnosis code contained within the electronic healthcare claim is used to make a determination of whether the event is qualifying or non-qualifying. In some implementations, if the event is determined to be a non-qualifying event, then the elect electronic healthcare claim is ignored or otherwise removed from consideration in making a frequent flyer determination. In some such implementations, process 300 may end (e.g., return to step 302 when a new electronic healthcare claim is obtained). Similarly, process 300 may end and/or return to step 302 if it is determined that the patient does not qualify as a frequent flyer.

If, however, the electronic healthcare claim is associated with a qualified event and it is determined that the patient qualifies as a frequent flyer (e.g., based at least on the number of visits to an ED/ER within a set time period), then process 300 may continue to step 312, where the patient is flagged as a frequent flyer. In some implementations, generating a flag includes generating or updating an electronic record associated with the patient. For example, the patient's electronic record can be updated and/or a new electronic record can be generated. The electronic record may identify the patient (e.g., by name), indicate that the patient qualifies as a frequent flyer, and provide further details regarding the rationale for the frequent flyer determination, such as a location of service (e.g., an address of the ED the patient most recently visited), a revenue code, diagnosis and/or procedure codes, payor identifier, and optionally patient demographic information. In some implementations, flagging the patient further or alternatively includes adding a patient identifier (e.g., the patient's name) to a list of frequent flyers.

In some implementations, before or after generating an alert, the patient can be assigned to a frequent flyer “category” based on the diagnosis/procedure codes contained in the electronic healthcare claim and the historical healthcare claims data. As mentioned above, frequent flyer categories may be predefined and can be used to categorize the patient based on the reason for their qualification as a frequent flyer (e.g., “substance abuse,” “chronic illness,” etc.). In some implementations, the assigned category is included with the flag, e.g., in the generated electronic record or appended to the patient's existing electronic record. In some implementations, an alert that identifies the patient and indicates that the patient qualifies as a frequent flyer is generated. In some implementations, the alert includes the assigned frequent flyer category. In some implementations, the alert is displayed to a user, such as a healthcare provider or healthcare payor representative, via a user interface or by transmitting the alert to the user's device.

At step 314, a care path (or “care management path) is optionally generated for the patient. As mentioned above, a care path generally includes one or more predicted future visits (e.g., with a healthcare provider, to a healthcare facility, or the like) for the patient to address the underlying reason for the patient being classified as a frequent flyer. In some implementations, the care path is determined based on the frequent flyer category assigned to the patient and/or based on the reason for the patient being classified as a frequent flyer. In some implementations, a plurality of care paths for common frequent flyer categories (e.g., drug use, alcohol abuse, etc.) are predefined. In some implementations, the care path includes at least one subsequent visit for the patient. In some implementations, the care path can be used to automatically schedule at least one subsequent visit with an appropriate healthcare professional (e.g., a counselor).

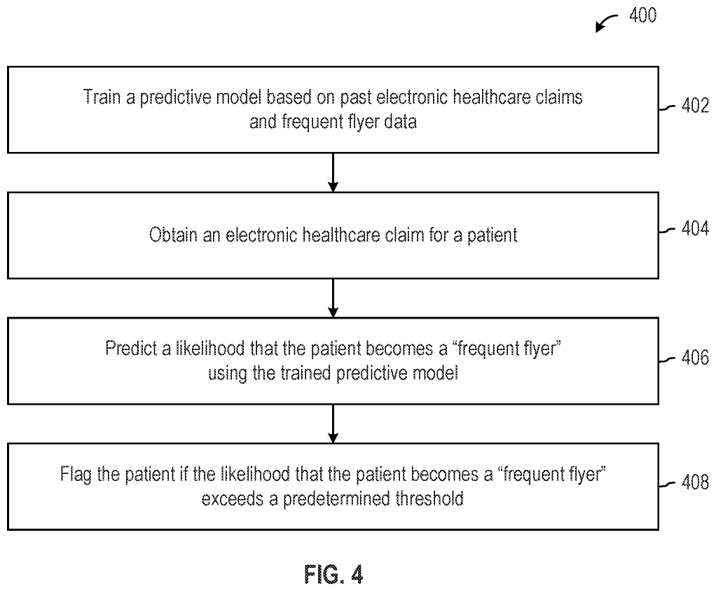

FIG. 4 is a flow diagram of a process 400 for predicting whether a patient is at risk of becoming a frequent flyer, according to some implementations. In some implementations, process 400 is implemented by system 200, as described above. However, it should be appreciated that process 400 may be implemented by other computing devices. For example, process 400 may be implemented via non-transitory computer readable medium or may be executed (e.g., as a script) on one of provider computers 102 or payor computers 104. It will be appreciated that certain steps of process 400 may be optional and, in some implementations, process 400 may be implemented using less than all of the steps. It will also be appreciated that the order of steps shown in FIG. 4 is not intended to be limiting.

At step 402, a predictive model is trained on past electronichealthcare claims and associated frequent flyer data. As described herein, the predictive model generally refers to a machine-learning model which is trained to predict a likelihood that the patient becomes a frequent flyer based on current and/or historical healthcare claims data. For example, the predictive model may be a neural network (e.g., an artificial neural network (ANN), a convolution neural network (CNN), etc.), a classifier (e.g., a logistic regression model, a Naive Bayes model, a K-nearest neighbors model, a decision tree, SVM, etc.), or the like. Generally, the predictive model is trained to output an indication of whether the patient is at risk of becoming a frequent flyer and/or a score that represents the likelihood that the patient becomes a frequent flyer based on input data extracted from an electronic healthcare claim.

It should be appreciated that the predictive model can generally be trained using any known supervised or unsupervised training techniques. In some implementations, the predictive model is trained using a training data set built on historic healthcare claims data (e.g., past electronic healthcare claims) for a plurality of patients, which includes service or receipt data, diagnosis and/or procedure codes, and the like for a plurality of electronic healthcare claims. In addition, the training data set generally includes an indication of whether each patient was found to be a frequent flyer, e.g., by system 200. For example, a training dataset can be constructed that includes past electronic healthcare claims for a plurality of different patient and that includes, for each patient, an indication of whether the patient qualifies as a frequent flyer and/or was flagged as a frequent flyer.

In some implementations, the training dataset is constructed from data collected by system 200, e.g., over time. For example, each time system 200 evaluates a new electronic healthcare claim, the extracted claims data and/or a copy of the claim can be stored, along with an indication of whether system 200 flagged the patient as a frequent flyer. In this manner, the training dataset can be built over time based on the flags generated by system 200. In other implementations, the training dataset can be manually constructed, e.g., by a person that manually evaluates historical healthcare claims data for a plurality of patients. In yet other implementations, the training dataset can be constructed by evaluating a batch of past electronic healthcare claims using system 200 to determine whether each of the associated patients qualifies as a frequent flyer (e.g., by applying flags as appropriate).

In some implementations, the predictive model is periodically updated or retrained as new frequent flyer data is obtained. In other words, the predictive model may be periodically retrained as system 200 evaluates new electronic healthcare claims. For example, each time system 200 evaluates an electronic healthcare claim to determine whether the associated patient qualifies as a frequent flyer, the results of the evaluation (e.g., “frequent flyer” or “not a frequent flyer”) may be recorded/stored along with a least a portion of the electronic healthcare claim data. The predictive model can then be retrained to include the new electronic healthcare claim(s) data and resulting frequent flyer determinations (e.g., flags) to improve accuracy in further predictions.

At step 404, an electronic healthcare claim is obtained for a patient. As described above, the electronic healthcare claim may be received directly from a healthcare provider and/or a medical biller or a healthcare payor. In some implementations, the electronic healthcare claim is intercepted as it is transmitted from a healthcare provider and/or medical biller to a healthcare payor. For example, electronic healthcare claims may pass through a clearinghouse that intercepts the claim. In some implementations, the electronic healthcare claim is retrieved from a database. For example, the electronic healthcare claim may be retrieved from a database associated with a healthcare payor or another remote database. In some implementations, the electronic healthcare claim is one of a batch of electronic healthcare claims that are intended to be processed together (e.g., sequentially). In some implementations, the electronic healthcare claim is maintained by a healthcare provider or payor, and process 300, as described herein, is implemented locally on the healthcare provider or payor's computing system.

At step 406, the predictive model is used to predict a likelihood that the patient becomes a frequent flyer. In particular, data from the electronic healthcare claim can be provided as an input to the predictive model, which outputs a corresponding prediction. In various implementations, the output is a class or label (e.g., “at risk” or “not at risk”), a binary indication (e.g., “yes” or “no”, ‘0’ or ‘1’), and/or a score (e.g., a percentage). In some implementations, as part of or prior to step 406, specific datapoints can be extracted from the electronic healthcare claim to be provided as the input to the predictive model. For example, the service and/or receipt data, diagnosis and/or procedure codes, and the like can be extracted to be provided as inputs. In some implementations, similar datapoints can also be extracted from the patient's historical healthcare claims data to be provided as inputs. In this way, the predictive model can use both historic and current healthcare claims data to predict whether the patient is likely to become a frequent flyer.

At step 408, the patient is flagged if the predicted likelihood that the patient becomes a frequent flyer exceeds a threshold. In some implementations, generating a flag includes generating or updating an electronic record associated with the patient. For example, the patient's electronic record can be updated and/or a new electronic record can be generated. Specifically, the patient's electronic record can be updated—or a new electronic record created—to indicate that the patient is likely to become a frequent flyer. In some implementations, an alert is generated which indicates that the patient is at risk of becoming a frequent flyer. In some implementations, the alert includes a frequent flyer category. In some implementations, the alert is displayed to a user, such as a healthcare provider or healthcare payor representative, via a user interface or by transmitting the alert to the user's device.

### Configuration of Certain Implementations

The construction and arrangement of the systems and methods as shown in the various exemplary implementations are illustrative only. Although only a few implementations have been described in detail in this disclosure, many modifications are possible (e.g., variations in sizes, dimensions, structures, shapes, and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the exemplary implementations without departing from the scope of the present disclosure.

The present disclosure contemplates methods, systems, and program products on any machine-readable media for accomplishing various operations. The implementations of the present disclosure may be implemented using existing computer processors, or by a special purpose computer processor for an appropriate system, incorporated for this or another purpose, or by a hardwired system. Implementations within the scope of the present disclosure include program products including machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures, and which can be accessed by a general purpose or special purpose computer or other machine with a processor.

When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general-purpose computer, special purpose computer, or special purpose processing machines to perform a certain function or group of functions.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps.

It is to be understood that the methods and systems are not limited to specific synthetic methods, specific components, or to particular compositions. It is also to be understood that the terminology used herein is for the purpose of describing particular implementations only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another implementation includes from the one particular value and/or to the other particular value. Similarly, when values are expressed as approximations, by use of the antecedent “about,” it will be understood that the particular value forms another implementation. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal implementation. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific implementation or combination of implementations of the disclosed methods.

What is claimed is:

1. A method of detecting patients who qualify as emergency department frequent flyers, the method comprising:
receiving an electronic healthcare claim;
obtaining historical healthcare claims data from an electronic record of a patient associated with the electronic healthcare claim;
determining whether the patient qualifies as an emergency department frequent flyer based on the electronic healthcare claim and the historical healthcare claims data, wherein an emergency department frequent flyer is defined as a patient with more than a threshold number of visits to an emergency department of one or more medical facilities within a time period; and
initiating responsive actions if it is determined that the patient qualifies as an emergency department frequent flyer, wherein the responsive actions include: i) generating a flag for the patient that identifies the patient as an emergency department frequent flyer, ii) assigning a frequent flyer category to the patient based on a procedure code or a diagnosis code contained in the electronic healthcare claim, and iii) modifying the electronic record of the patient to include the flag and the frequent flyer category.

2. The method of claim 1, wherein the responsive actions further include:
generating a care management path for the patient, wherein the care management path defines at least one subsequent visit with a medical professional to address a cause of the patient qualifying as an emergency department frequent flyer.

3. The method of claim 1, wherein the responsive actions further include:
generating an alert that identifies the patient as an emergency department frequent flyer; and
presenting the alert to a user.

4. The method of claim 3, wherein presenting the alert to the user includes:
identifying a recipient for the alert based on the electronic healthcare claim; and
transmitting the alert to a remote computing device associated with the recipient.

5. The method of claim 1, further comprising:
determining whether the electronic healthcare claim contains one or more procedure or diagnosis codes associated with a non-qualifying event, wherein the electronic healthcare claim is excluded from consideration in determining whether the patient qualifies as an emergency department frequent flyer if it is determined that the electronic healthcare claim is associated with a non-qualifying event.

6. The method of claim 1, further comprising extracting, from the electronic healthcare claim:
a service date associated with the electronic healthcare claim or a receipt date of the electronic healthcare claim, an identifier for a medical facility at which the patient was treated, a revenue code, a procedure code or a diagnosis code, a filing indicator code, and a payor identifier.

7. The method of claim 1, wherein the threshold number of visits to an emergency department of one or more medical facilities within a time period comprises either:
three visits within three months; or
four visits within twelve months.

8. The method of claim 1, wherein the electronic healthcare claim is one of a batch of electronic healthcare claims for a plurality of patients, wherein the method is repeated for each claim of the batch of electronic healthcare claims to determine whether any of the plurality of patients qualify as emergency department frequent flyers.

9. The method of claim 1, wherein receiving the electronic healthcare claim comprises intercepting the electronic healthcare claim as it is transmitted from a healthcare provider computer to a healthcare payor computer.

10. A system for detecting patients who qualify as emergency department frequent flyers, the system comprising:
one or more processors; and
memory having instructions stored thereon that, when executed by the one or more processors, cause the system to:
receive an electronic healthcare claim;
obtain historical healthcare claims data from an electronic record of a patient associated with the electronic healthcare claim;
determine whether the patient qualifies as an emergency department frequent flyer based on the electronic healthcare claim and the historical healthcare claims data, wherein an emergency department frequent flyer is defined as a patient with more than a threshold number of visits to an emergency department of one or more medical facilities within a time period; and
initiate responsive actions if it is determined that the patient qualifies as an emergency department frequent flyer, wherein the responsive actions include: i) generating a flag for the patient that identifies the patient as an emergency department frequent flyer, ii) assigning a frequent flyer category to the patient based on a procedure code or a diagnosis code contained in the electronic healthcare claim, and iii) modifying the electronic record of the patient to include the flag and the frequent flyer category.

11. The system of claim 10, wherein the responsive actions further include:
generating a care management path for the patient, wherein the care management path defines at least one subsequent visit with a medical professional to address a cause of the patient qualifying as an emergency department frequent flyer.

12. The system of claim 10, wherein the responsive actions further include:
generating an alert that identifies the patient as an emergency department frequent flyer; and
presenting the alert to a user.

13. The system of claim 12, wherein presenting the alert to the user includes to:
identifying a recipient for the alert based on the electronic healthcare claim; and
transmitting the alert to a remote computing device associated with the recipient.

14. The system of claim 10, wherein the instructions further cause the system to:
determining whether the electronic healthcare claim contains one or more procedure or diagnosis codes associated with a non-qualifying event, wherein the electronic healthcare claim is excluded from consideration in determining whether the patient qualifies as an emergency department frequent flyer if it is determined that the electronic healthcare claim is associated with a non-qualifying event.

15. The system of claim 10, wherein the instructions further cause the system to extract, from the electronic healthcare claim:
a service date associated with the electronic healthcare claim or a receipt date of the electronic healthcare claim, an identifier for a medical facility at which the patient was treated, a revenue code, a procedure code or a diagnosis code, a filing indicator code, and a payor identifier.

16. The system of claim 10, wherein the threshold number of visits to an emergency department of one or more medical facilities within a time period comprises either:
three visits within three months; or
four visits within twelve months.

17. The system of claim 10, wherein the electronic healthcare claim is one of a batch of electronic healthcare claims for plurality of patients, wherein each claim of the batch of electronic healthcare claims is evaluated to determine whether any of the plurality of patients qualify as emergency department frequent flyers.

18. The system of claim 10, wherein receiving the electronic healthcare claim comprises intercepting the electronic healthcare claim as it is transmitted from a healthcare provider computer to a healthcare payor computer.

19. A non-transitory computer readable medium having instructions stored thereon that, when executed by one or more processors, cause a device to:
receive an electronic healthcare claim;
obtain historical healthcare claims data from an electronic record of a patient associated with the electronic healthcare claim;
determine whether the patient qualifies as an emergency department frequent flyer based on the electronic healthcare claim and the historical healthcare claims data, wherein an emergency department frequent flyer is defined as a patient with more than a threshold number of visits to an emergency department of one or more medical facilities within a time period; and
initiate responsive actions if it is determined that the patient qualifies as an emergency department frequent flyer, wherein the responsive actions include: i) generating a flag for the patient that identifies the patient as an emergency department frequent flyer, ii) assigning a frequent flyer category to the patient based on a procedure code or a diagnosis code contained in the electronic healthcare claim, and iii) modifying the electronic record of the patient to include the flag and the frequent flyer category.

20. The computer readable medium of claim 19, wherein the responsive actions further include:
generating a care management path for the patient, wherein the care management path defines at least one subsequent visit with a medical professional to address a cause of the patient qualifying as an emergency department frequent flyer.
